(19) United States
(12) Patent Application Publication
(10) Pub. No.: US 2013/0165811 A1
(43) Pub. Date: Jun. 27, 2013

(54) APPARATUS FOR AUTOMATICALLY DIAGNOSING EMPHYSEMA

(71) Applicants: Marc Decramer, Pellenberg (BE); Wim Janssens, Kessel-Lo (BE)

(72) Inventors: Marc Decramer, Pellenberg (BE); Wim Janssens, Kessel-Lo (BE)

(21) Appl. No.: 13/820,375

(22) PCT Filed: Sep. 1, 2011
(86) PCT No.: PCT/BE2011/000055

§ 371 Date: Mar. 1, 2013

(30) Foreign Application Priority Data
Sep. 2, 2010 (GB) 1014588.6
Feb. 14, 2011 (GB) 1102525.1
Feb. 14, 2011 (GB) 1102527.7
Feb. 14, 2011 (GB) 1102528.5

(51) Int. Cl. A61B 5/08

(52) U.S. Cl. CPC A61B 5/0803
USPC 600/538

(57) ABSTRACT

The present invention relates generally to diagnosing chronic obstructive pulmonary disease (COPD) and estimating of the severity thereof, in particularly the occurrence of emphysema. More particularly the present invention relates to a system and method for computerized quantification of airway collapse during forced expiration or total exhalation. Such airway collapse is correlated with the presence of emphysema that has been verified on computer tomography (CT) scan and that is due to the loss of alveolar attachments in emphysema. Moreover the present invention provides a computerized apparatus for detection of emphysema, a computerized system for detection of emphysema or a method of computer-aided detection of emphysema. This can also be used for quantifying the severity of emphysema in patients with COPD by an automated analysis of measurement of the amount (volume) and/or speed (flow) of a total or forced exhalation or the processing of the graphics of a pneumotachograph of a total or forced exhalation. Such measurements are obtainable by a spirometer. The apparatus, system or method of present invention correlates airway collapse during forced expiration in COPD with presence and severity of emphysema It was demonstrated that airway collapse during forced expiration could be quantified by measuring the angle between the two best fitting regression lines describing the cloud of point measurements obtained from peakflow to the end of forced expiration. Such angle in COPD correlates with presence and severity of emphysema, as assessed by computer tomography scan.

### BACKGROUND AND SUMMARY

### Background of the Invention

A. Field of the Invention

The present invention relates generally to diagnosing chronic obstructive pulmonary disease (COPD) and estimating of the severity thereof, in particularly the occurrence of emphysema. More particularly the present invention relates to a system and method for computerized quantification of airway collapse during forced expiration or total exhalation. Such airway collapse is correlated with the presence of emphysema that has been verified on computer tomography (CT) scan and that is due to the loss of alveolar attachments in emphysema.

Moreover the present invention provides a computerized apparatus for detection of emphysema, a computerized system for detection of emphysema or a method of computer-aided detection of emphysema. This can also be used for quantifying the severity of emphysema in patients with COPD by an automated analysis of the amount (volume) and/or speed (flow) of a total or forced exhalation or the processing of the graphics of a pneumotachograph of a total or forced exhalation. Such measurements are obtainable by a spirometer.

The apparatus, system or method of present invention correlates airway collapse during forced expiration in COPD with presence and severity of emphysema. It was demonstrated that airway collapse during forced expiration in COPD correlates with presence and severity of emphysema.

Several documents are cited throughout the text of this specification. Each of the documents herein (including any manufacturer's specifications, instructions etc.) are herby incorporated by reference; however, there is no admission that any document cited is indeed prior art of the present invention.

B. Description of the Related Art

Worldwide, COPD ranked as the sixth leading cause of death in 1990. It is projected to be the fourth leading cause of death worldwide by 2030 due to an increase in smoking rates and demographic changes in many countries. COPD is the 4th leading cause of death in the U.S., and the economic burden of COPD in the U.S. in 2007 was $42.6 billion in health care costs and lost productivity. The diagnosis of COPD requires lung function tests such as respirometry.

Emphysema is a long-term, progressive disease of the lung. It is included in a group of diseases called COPD. Emphysema is called an obstructive lung disease because the destruction of lung tissue around smaller airways, called alveoli, makes these airways unable to hold their functional shape upon exhalation.

Forced expiratory volumes and flows or the expiratory volumes and flows of total exhalation are used to diagnose COPD. However the current methods and systems in the art do not accurately distinguish airway obstruction from emphysema and are thus not suitable for accurate distinguishing emphysema from COPD and for estimating the severity of emphysema.

Thus, there is a need in the art for more accurate diagnosis systems and methods to detect emphysema and assess the severity of emphysema in a group of diseases called COPD.

Present invention provides such by computerized quantification of airway collapse during forced expiration and demonstrates correlations with the presence of emphysema on CT scan.

### SUMMARY OF THE INVENTION

The present invention solves the problems of the related art of inaccurate diagnosing of emphysema by providing an apparatus for diagnosing emphysema, the apparatus comprising a) a device for receiving and sensing forced or total expiration of the respiratory system of a patient and further comprising b) a calculator or signal processor calculating the flow volume curve or relationship from said forced or total airway expiration, characterized in that the calculator or signal processor comprises 1) a first calculating means for calculating the flow-volume curve or the flow-volume relationship and 2) a second calculating means for automatically calculating the angle in the expiratory flow-volume curve between the two best fitting linear regression curves on flow-volume curve.

In accordance with the purpose of the invention, as embodied and broadly described herein, the invention is broadly drawn to an apparatus for diagnosing emphysema whereby the apparatus having a) a signal input to receive electrical signals of an electrical signals produced by volume sensor and flow sensors of a device for receiving and sensing forced or total expiration of the respiratory system of a patient and b) a calculator or signal processor calculating the flow volume curve or relationship from said forced or total airway expiration, characterized in that the calculator or signal processor comprises 1) a first calculating means for calculating the flow-volume curve or the flow-volume relationship and 2) a second calculating means for automatically calculating the angle in the expiratory flow-volume curve between the two best fitting linear regression curves on the flow-volume curve corresponding to entire exhalation flow rate and exhalation volume of a patients respiratory system.

This apparatus of present invention or described above can further comprise calculating means for calculating the peak-to-surface area. Moreover in the apparatus of present invention or as described above the signal processor or calculator further can comprise a mathematical model to compare said angle with that of a plurality of chronic obstructive pulmonary disease (COPD) patients with no emphysema. Moreover the signal processor or calculator further comprising a mathematical model to compare said angle with that of a plurality of COPD patients affected with emphysema. In yet another embodiment the signal processor or calculator further comprises a mathematical model to compare said angle with that of a plurality of COPD patients affected with a defined seriousness or with defined progress of emphysema. Furthermore the signal processor or calculator in the apparatus here above described can further comprise a mathematical model to compare said angle with that of a control or the signal processor comprises a mathematical model that is described to automatically calculate expiratory airway collapse due to loss of alveolar attachments in emphysema.

The device for receiving and sensing forced or total expiration of the respiratory system of a patient can be for present invention a spirometer.

Furthermore the present invention concerns the use of the apparatus of any one of the embodiments described here above to define emphysema or for diagnosing emphysema; to define the seriousness or progress of emphysema or to define a respiratory therapy medication. This is a first time accurate diagnosing of emphysema without radiological imaging such as CT scanning.

Another embodiment of present invention is a system for diagnosing an emphysema disorder in a chronic obstructive pulmonary disease (COPD) patient, the system comprising: 1) a sampling device to obtain total expiration or exhalation of the respiratory system of the subject; 2) a detection device generating flow and volume data of said total expiration or exhalation; and 3) a computer loaded model to calculate the flow-volume loop; the two best fitting linear regression curves on flow-volume loop from peak flow to end of expiration and the angle between both regression lines and further loaded with a flow-volume loop reference profile for a emphysema disorder; wherein the computer receives and compares subject's flow-volume loop profile with the reference flow-volume loop profile.

Yet another embodiment of present invention is a system for diagnosing an emphysema disorder in a chronic obstructive pulmonary disease (COPD) patient, the system comprising: a sampling device to obtain total expiration or exhalation of the respiratory system of the subject; a detection device generating flow and volume data of said total expiration or exhalation; and a computer loaded model to calculate the flow-volume loop; the two best fitting linear regression curves on flow-volume loop from peak flow to end of expiration and the angle between both regression lines and further loaded with a flow-volume loop reference profile for an emphysema disorder; wherein the computer receives and compares subject's flow-volume loop profile with the reference flow-volume loop profile.

Another aspect of the invention is a method of diagnosing an emphysema disorder in a subject with COPD, the method comprising: generating flow and volume data of total expiration or forced exhalation of a subject, calculating a profile of the two best fitting linear regression curves on flow-volume loop from peak flow and the angle between both regression lines, thereby providing; obtaining a reference profile for emphysema or for no emphysema; and comparing the subject profile with the reference profile, wherein a match of the subject profile to the reference profile indicates that the subject has emphysema or has no emphysema.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description. It is to be understood that both the foregoing general description and the following detailed description are exemplary and explanatory only and are not restrictive of the invention, as claimed.

The present invention relates to an apparatus for diagnosing emphysema, the apparatus comprising a) a device for receiving and sensing forced or total expiration of the respiratory system of a patient and further comprising b) a calculator or signal processor calculating the flow volume curve or relationship from said forced or total airway expiration, characterized in that the calculator or signal processor comprises 1) a first calculating means for calculating the flow-volume curve or the flow-volume relationship and 2) a second calculating means for automatically calculating the angle in the expiratory flow-volume curve between the two best fitting linear regression curves on flow-volume curve. This apparatus can further comprise a calculating means for calculating the peak-to-surface area. The object of the present invention is also a an apparatus for diagnosing emphysema whereby the apparatus having a) a signal input to receive electrical signals of an electrical signals produced by volume sensor and flow sensors of a device for receiving and sensing forced or total expiration of the respiratory system of a patient and b) a calculator or signal processor calculating the flow volume curve or relationship from said forced or total airway expiration, characterized in that the calculator or signal processor comprises 1) a first calculating means for calculating the flow-volume curve or the flow-volume relationship and 2) a second calculating means for automatically calculating the angle in the expiratory flow-volume curve between the two best fitting linear regression curves on the flow-volume curve corresponding to entire exhalation flow rate and exhalation volume of a patients respiratory system. This apparatus can further comprise a calculating means for calculating the peak-to-surface area.

With respect to the signal processor or calculator, it is noted that it is advantageous if this signal processor or calculator further comprising a mathematical model to compare said angle with that of plurality of chronic obstructive pulmonary disease (COPD) patients with no emphysema. A further disadvantageous aspect is also, that the signal processor or calculator further comprising a mathematical model to compare said angle with that of plurality of COPD patients affected with emphysema or that the signal processor or calculator further comprising a mathematical model to compare said angle with that of plurality of COPD patients affected with a defined seriousness or with defined progress of emphysema. In a particular embodiment the signal processor or calculator in these apparatus of present invention further comprises a mathematical model to compare said angle with that of a control. In yet another embodiments these apparatuses are characterized in that the apparatus comprises a signal processor comprising a mathematical model that is described to automatically calculate expiratory airway collapse due to loss of alveolar attachments in emphysema.

In an advantageous embodiment, the apparatuses according to the present invention further comprises a device for receiving and sensing forced or total expiration of the respiratory system of a patient is a spirometer.

The object of the present invention is also to provide a use of the apparatuses according to the present invention to define emphysema or for diagnosing emphysema.

The use of the apparatuses according to the present invention van be for any of the following: to define the seriousness or progress of emphysema; to define a respiratory therapy medication; to define emphysema or for diagnosing emphysema without radiological imaging such as CT scanning; to define emphysema or for diagnosing emphysema whereby an angle smaller than 140° is indicative for emphysema; to define emphysema or for diagnosing emphysema whereby an angle smaller than 135° is indicative for emphysema; to define emphysema or for diagnosing emphysema whereby an angle smaller than 130° is indicative for emphysema; to define emphysema or for diagnosing emphysema whereby an angle smaller than 125° is indicative for emphysema or to define emphysema or for diagnosing emphysema whereby an angle smaller than 120° is indicative for emphysema.

Another object of the present invention is to provide a system for diagnosing a emphysema disorder in a chronic obstructive pulmonary disease (COPD) patient, the system can comprise: 1) a sampling device to obtain total expiration or exhalation of the respiratory system of the subject; 2) a detection device generating flow and volume data of said total expiration or exhalation; and 3) a computer loaded a model a model to calculate the flow-volume loop; the two best fitting linear regression curves on flow-volume loop from peak flow to end of expiration and the angle between both regression lines and further loaded with a flow-volume loop reference profile for a emphysema disorder; wherein the computer receives and compares subject's flow-volume loop profile with the reference flow-volume loop profile or the system can comprise a sampling device to obtain total expiration or exhalation of the respiratory system of the subject; a detection device generating flow and volume data of said total expiration or exhalation; and a computer loaded model to calculate the flow-volume loop; the two best fitting linear regression curves on flow-volume loop from peak flow to end of expiration and the angle between both regression lines and further loaded with a flow-volume loop reference profile for an emphysema disorder; wherein the computer receives and compares subject's flow-volume loop profile with the reference flow-volume loop profile.

Yet another object of the present invention is to provide to provide method of diagnosing an emphysema disorder in a subject with COPD, the method comprising: generating flow and volume data of total expiration or forced exhalation of a subject, calculating a profile of the two best fitting linear regression curves on flow-volume loop from peak flow and the angle between both regression lines, thereby providing; obtaining a reference profile for emphysema or for no emphysema; and comparing the subject profile with the reference profile, wherein a match of the subject profile to the reference profile indicates that the subject has emphysema or has no emphysema.

Some embodiments of the invention are set forth in claim format directly below:

### DETAILED DESCRIPTION

### Detailed Description of Embodiments of the Invention

The following detailed description of the invention refers to the accompanying drawings. The same reference numbers in different drawings identify the same or similar elements. Also, the following detailed description does not limit the invention. Instead, the scope of the invention is defined by the appended claims and equivalents thereof.

Several documents are cited throughout the text of this specification. Each of the documents herein (including any manufacturer's specifications, instructions etc.) are hereby incorporated by reference; however, there is no admission that any document cited is indeed prior art of the present invention.

The present invention will be described with respect to particular embodiments and with reference to certain drawings but the invention is not limited thereto but only by the claims. The drawings described are only schematic and are non-limiting. In the drawings, the size of some of the elements may be exaggerated and not drawn to scale for illustrative purposes. The dimensions and the relative dimensions do not correspond to actual reductions to practice of the invention.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

Moreover, the terms top, bottom, over, under and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

It is to be noticed that the term “comprising”, used in the claims, should not be interpreted as being restricted to the means listed thereafter; it doe not exclude other elements or steps. It is thus to be interpreted as specifying the presence of the stated features, integers, steps or components as referred to, but doe not preclude the presence or addition of one or more other features, integers, steps or components, or groups thereof. Thus, the scope of the expression “a device comprising means A and B” should not be limited to the devices consisting only of components A and B. It means that with respect to the present invention, the only relevant components of the device are A and B.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the description of exemplary embodiments of the invention, various features of the invention are sometimes grouped together in a single embodiment, figure, or description thereof for the purpose of streamlining the disclosure and aiding the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

In the description provided herein, numerous specific details are set forth. However, it is understood that embodiments of the invention may be practiced without these specific details. In other instances, well-known methods, structures and techniques have not been shown in detail in order not to obscure an understanding of this description.

Forced expiratory volumes and flows are used to diagnose COPD and estimate the severity thereof They do not accurately distinguish airway obstruction from emphysema. We investigated whether a computerized detection and quantification of airway collapse during forced expiration and measurement of the amount (volume) and/or speed (flow) of a total or forced exhalation correlated with the presence of emphysema on CT scan. The amount (volume) and/or speed (flow) of a total or forced exhalation can be assessed by a spirometer. A spirometer is an apparatus for measuring the volume of air inspired and expired by the lungs. It is a precision differential pressure transducer for the measurements of respiration flow rates. The spirometer records the amount of air and the rate of air that is breathed in and out over a specified period of time. An incentive spirometer is used to help patients improve the functioning of their lungs. Tank-type spirometer works as the same principle as the gasometer. A canister of soda is usually attached to absorb carbon dioxide and a kymograph trace is produced to record changes in total volume gas. From this, vital capacity, tidal volume, breathing rate and ventilation rate (=tidal volume×breathing rate) can be calculated. From the overall decline on the graph, the oxygen uptake can also be measured

### EXAMPLES

### Example 1

513 patients with >15 pack-years and >50 years were enrolled. Electronic data of the best spirometry (ATS/ERS criteria) were used to calculate the two best fitting linear regression curves on flow-volume loops from peak flow to end of expiration. The angle between both regression lines (AC) was used as surrogate marker of airway collapse. AC was related to diffusing capacity over alveolar ventilation (KCO), another functional variable known to be associated with emphysema but NOT accessible with spirometry in a general practise. AC was also related to semi-quantitative visual scores of emphysema on CT.

In 93% of patients (n=477) the computer model resulted in a correct quantification of mean AC, 156°±7° in healthy subjects (n=138) and 135°±10° in COPD patients (n=339). In subjects with FEV1/FVC ratio >0.7, ACs were not different between emphysema and non-emphysema subgroups. In COPD patients however, the mean AC in the emphysema subgroup (n=238) was significantly lower as compared to the non-emphysema group (n=101) (130° vs. 145°), even when stratifying for GOLD stage (p<0.0001). Multivariate analysis retained KCO as the best indicator of emphysema on CT scan. When considering only spirometry, AC was clearly the best predictor of visually scored emphysema (R2=0.43, p<0.0001), whereas other variables such as FEV1, FEV1/FVC ratio, time of expiration, peak flow to surface ratio and SVC-VC difference did not further contribute to the regression model. Finally, receiver operating curves defined 130° as best cut-off for emphysema with a specificity of 95% and sensitivity of 52% (AUC=0.82, p<0.0001).

Airway collapse on expiratory flow reflected by AC, correlates with severity of emphysema. A cut-off of 130° may identify emphysema in general practice with high certainty. Other embodiments of the invention will be apparent to those skilled in the art from consideration of the specification and practice of the invention disclosed herein. It is intended that the specification and examples be considered as exemplary only. Each and every claim is incorporated into the specification as an embodiment of the present invention. Thus, the claims are part of the description and are a further description and are in addition to the preferred embodiments of the present invention. Each of the claims set out a particular embodiment of the invention. The following terms are provided solely to aid in the understanding of the invention.

### Example 2

### A Calculation Process

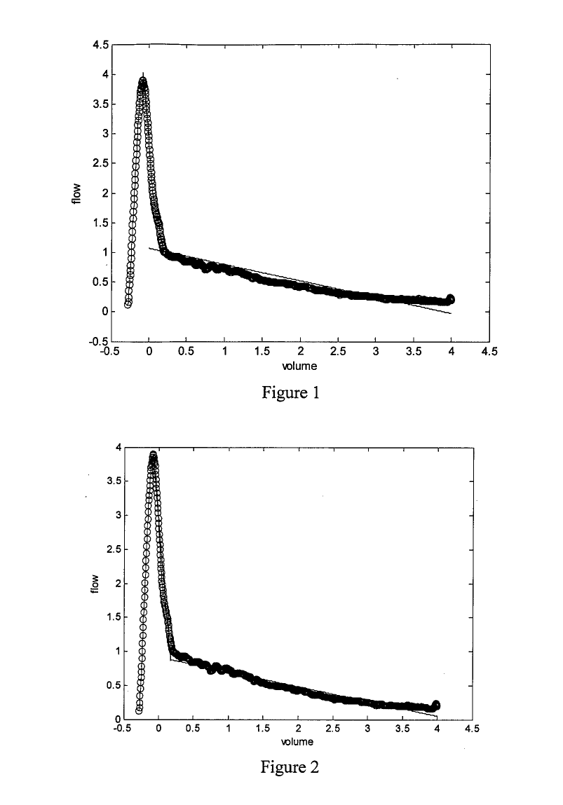

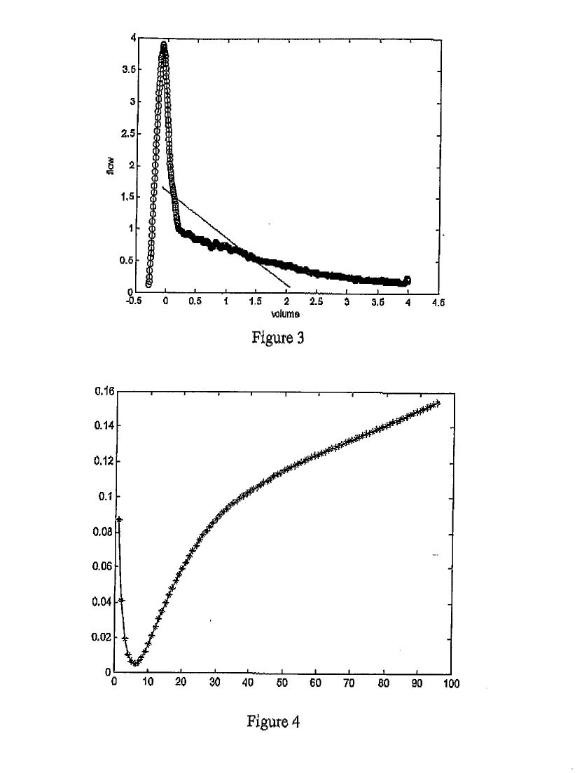

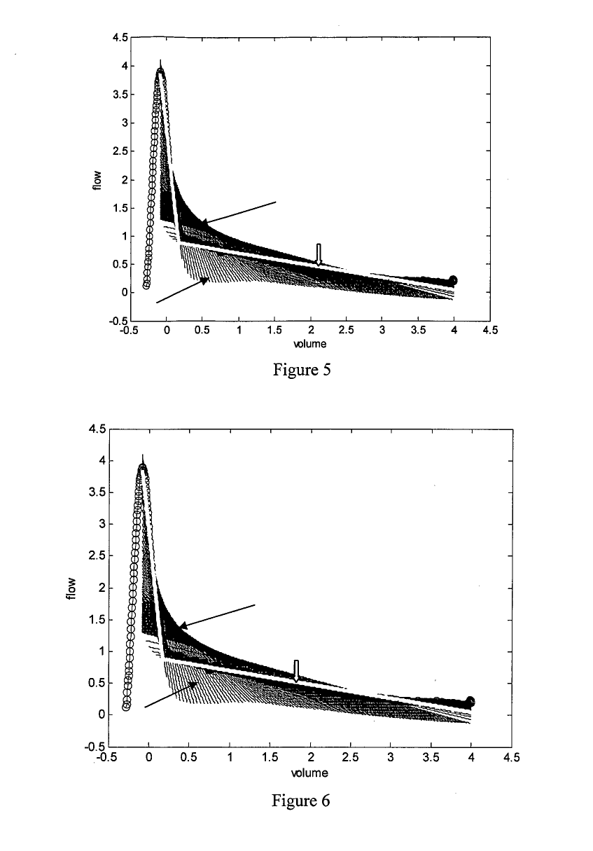

As first COPD calculator the peak-to-surface value of flow-volume curve or the flow-volume relationship. The peak-to-surface value is the peak value divided by the surface under the measured curve. As second COPD indicator the angle between two lines is calculated, which lines describe the point cloud between the peak and the maximal volume in the meaning that the mean square error between the data and fitted curve are minimal. Since it is initially not known which angle points to select or which portion of the data to fit with curve one and which of the remaining portion to fit with curve 2, the automated program makes different runs. For each run the automated program selects a new angle point with a 10 points interval starting at peakflow. For instance 1) for the first run (run 1) we select the angle point 1×10 (=10 samples) after the peak and a fit run is carried out 2) for the second run (run 2) an angle point is selected at 2×10 (=20 samples) of the peak and a fit is carried out and 3) such operations with the same or similar sample gaps are repeated. Such examples are demonstrated in the FIGS. 1-3. FIG. 1 demonstrates the best fitting linear regression curves at run 2 (the angle point at 2×10 (=20 samples) of the peak. The fit is acceptable but not yet optimal. FIG. 2 demonstrates the fit for the repetitive runs at run 6 (the angle point being selected at 6×10 (=60 samples) of the peak. This already demonstrates a best fit. FIG. 3 demonstrates a fit for run 50; i.e. if we select an angle point at 50×10 (=500 samples) of the peak. Here the fit is suboptimal. FIG. 4 demonstrates the goodness of fit of all runs and shows the mean square error for al the runs. Hereby is clearly demonstrated that the best total fit occurred at run 6 (angle point selected at 6×10 (=60 samples) of the peak. An overview of all runs is provided in FIG. 5. FIG. 5 demonstrates the fits for all runs (run 1 to run 5000) which is designate by the single line arrow) →).The fit of the best run is designated by double line arrow [symbol] ). this concerns run six with angular point at 6*10 =60 sample points of the peak. The MSE (mean square error) was hereby minimal This FIG. 5 demonstrates the calculated two best fitting linear regression curves on flow-volume loops from peak flow to end of expiration and the angle between both regression lines (AC).

Particular and preferred aspects of the invention are set out in the accompanying independent and dependent claims. Features from the dependent claims may be combined with features of the independent claims and with features of other dependent claims as appropriate and not merely as explicitly set out in the claims. Thus, the claims following the detailed description are hereby expressly incorporated into this detailed description, with each claim standing on its own as a separate embodiment of this invention.

### DRAWING DESCRIPTION

### Brief Description of the Drawings

The present invention will become more fully understood from the detailed description given herein below and the accompanying drawings which are given by way of illustration only, and thus are not limitative of the present invention, and wherein:

FIG. 1: concerns a COPD analysis by the measurement of the amount (volume) and/or speed (flow) of air of a forced exhalation and displays the fit through two linear regression line (defining a relationship between the flow and the volume) on 20 samples points of the peak of the flow volume curve from said forced airway expiration.

FIG. 2: concerns a COPD analysis by the measurement of the amount (volume) and/or speed (flow) of air of a forced exhalation and displays the fit through two linear regression line (defining a relationship between the flow and the volume) on 60 samples points of the peak of the flow volume curve from said forced airway expiration.

FIG. 3: concerns a COPD analysis by the measurement of the amount (volume) and/or speed (flow) of air of a forced exhalation and displays the fit through two linear regression line (defining a relationship between the flow and the volume) on 500 samples points of the peak of the flow volume curve from said forced airway expiration.

FIG. 4: displays the MSE (mean square error) of different runs. A minimums is reached at run 6, e.g. if we select an angular point on 6*10=60 sampling points from the peak. Bij dit minimum hebben we m.a.w. de beste globale fit.

FIG. 5: demonstrates the fits for all runs (designate by the single line arrow →). The fit of the best run is designated by double line arrow [symbol] ). this concerns run six with angular point at 6*10=60 sample points of the peak. The MSE (mean square error) was hereby minimal. This figure demonstrates the calculated two best fitting linear regression curves on flow-volume loops from peak flow to end of expiration and the angle between both regression lines (AC)

FIGS. 6 to 15 concerns measurement of the amount (volume) and/or speed (flow) (designate by the single line arrow →). of air of a forced exhalation and displays the fit on 10 different patients. These figures demonstrate the calculated two best fitting linear regression curves (designated by double line arrow [symbol] ) on flow-volume loops from peak flow to end of expiration and the angle between both regression lines (AC)

FIG. 6: Peak_to_surface=1.3893 and Angle=109.4244

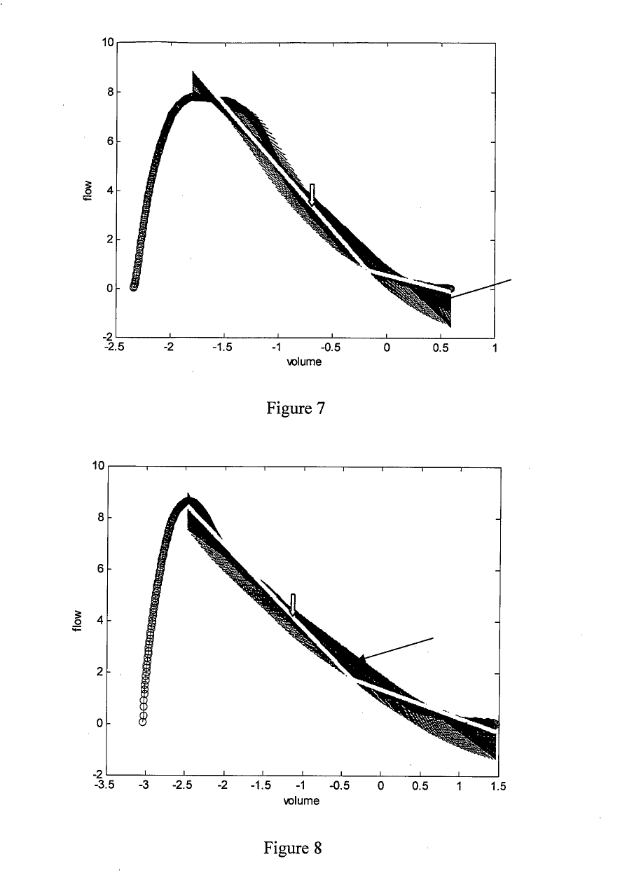

FIG. 7: Peak_to_surface=0.7073 and Angle=154.0281

FIG. 8: Peak_to_surface: 0.5547 and Angle: 154.5251

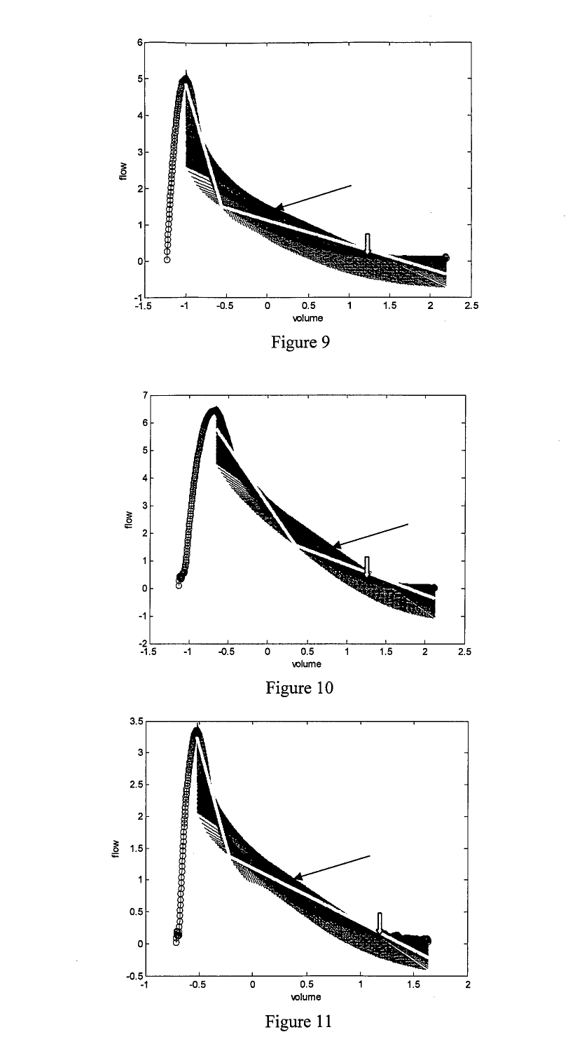

FIG. 9: Peak_to_surface: 1.3299 and Angle: 132.5744

FIG. 10: Peak_to_surface: 0.9722 and Angle: 149.8245

FIG. 11: Peak_to_surface: 1.5545 and Angle: 140.2217

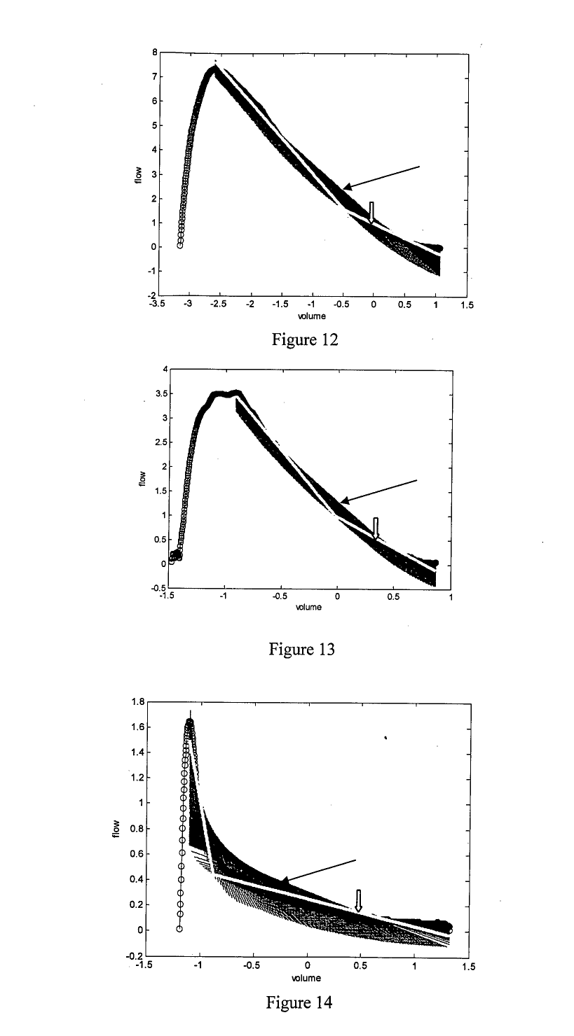

FIG. 12: Peak_to_surface: 0.5386 and Angle: 159.2410

FIG. 13: Peak_to_surface: 0.9283 and Angle: 158.3582

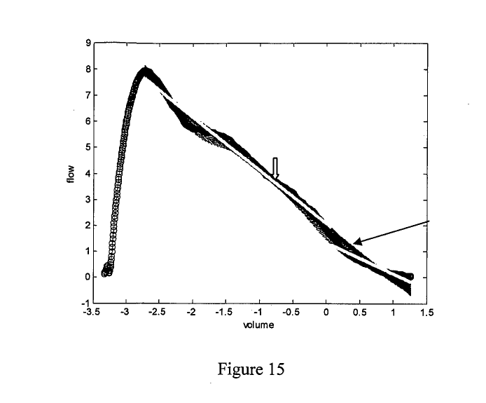

FIG. 14: Peak_to_surface: 2.1694 and Angle: 117.7351

FIG. 15: Peak_to_surface: 0.4691 and Angle: 166.5198

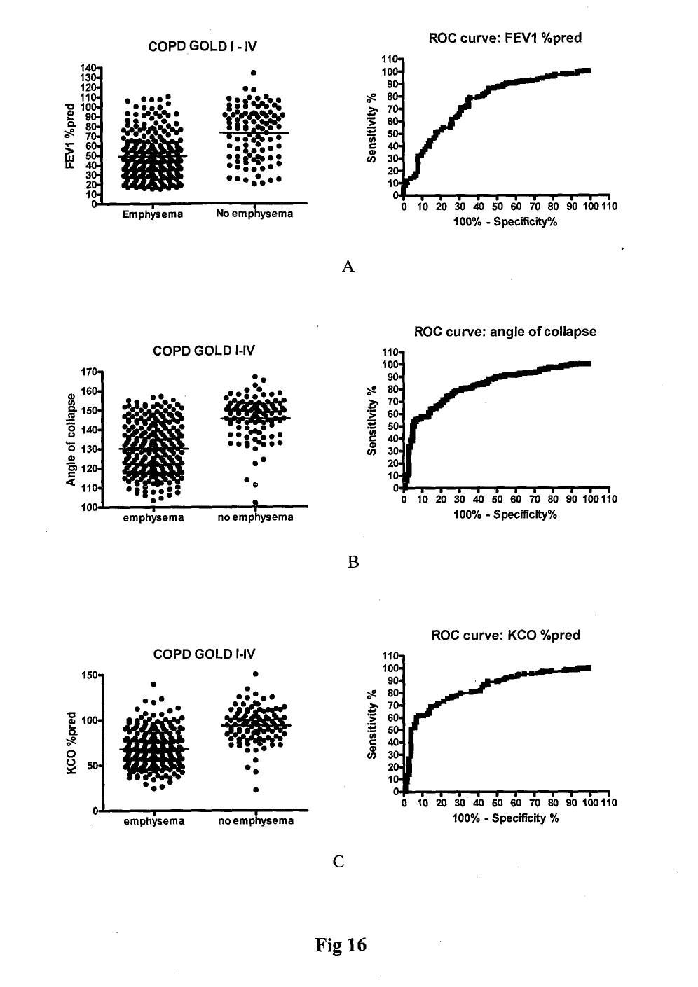

FIG. 16a-c provides an overview of COPD analysis on the patient as described in example 1. The receiver operating characteristic demonstrated that 130° provides the best cut off value to diagnose emphysema in the flow-volume curve that represents the measurement of the amount (volume) and/or speed (flow) of air of a forced exhalation of a COPD patient (FIG. 16a). The specificity was 95%. The sensitivity was 52% (area under the curve AUC=0.82, p<0.0001) There are none or very few patients with an angle smaller than 130° who do not have the emphysema disorder as could be confirmed via a visual score system on CT-images. FIG. 16b demonstrates that lower FEV1 % relates to more patients with emphysema but is also common in COPD patients without emphysema and can therefore not be used for discrimination. FIG. 16c demonstrates that diffusing capacity corrected for alveolar volume, a previously accepted non spirometry-derived predictor for emphysema, is almost equally efficient as the angle of collaps, a variable which can be obtained from every spirometry.

Accordingly, the present invention provides a system for diagnosing a emphysema disorder in a chronic obstructive pulmonary disease (COPD) patient, the system comprising: 1) a sampling device to obtain total expiration or exhalation of the respiratory system of the subject; 2) a detection device generating flow and volume data of said total expiration or exhalation; and 3) a computer loaded a model a model to calculate the flow-volume loop; the two best fitting linear regression curves on flow-volume loop from peak flow to end of expiration and the angle between both regression lines and further loaded with a flow-volume loop reference profile for a emphysema disorder; wherein the computer receives and compares subject's flow-volume loop profile with the reference flow-volume loop profile. The present invention also provides a system for diagnosing a emphysema disorder in a chronic obstructive pulmonary disease (COPD) patient, the system comprising: a sampling device to obtain total expiration or exhalation of the respiratory system of the subject; a detection device generating flow and volume data of said total expiration or exhalation; and a computer loaded model to calculate the flow-volume loop; the two best fitting linear regression curves on flow-volume loop from peak flow to end of expiration and the angle between both regression lines and further loaded with a flow-volume loop reference profile for an emphysema disorder; wherein the computer receives and compares subject's flow-volume loop profile with the reference flow-volume loop profile.

In one embodiment of the invention, a method of diagnosing an emphysema disorder in a subject with COPD is provided whereby the method comprising: generating flow and volume data of total expiration or forced exhalation of a subject, calculating a profile of the two best fitting linear regression curves on flow-volume loop from peak flow and the angle between both regression lines, thereby providing; obtaining a reference profile for emphysema or for no emphysema; and comparing the subject profile with the reference profile, wherein a match of the subject profile to the reference profile indicates that the subject has emphysema or has no emphysema.

This invention accordingly provides the advantage that emphysema disorder can be distinguished in a in a chronic obstructive pulmonary disease (COPD) patient group.

In an advantageous embodiment, the method or system according to the present invention and described here above comprise a calculating means for calculating the peak-to-surface area. In any of the different embodiments it comprises a mathematical model that compares said angle with that of plurality of chronic obstructive pulmonary disease (COPD) patients with no emphysema. Hereby the mathematical model can compare the angle with that of plurality of COPD patients affected with emphysema; the mathematical model can compare said angle with that of plurality of COPD patients affected with a defined seriousness or with defined progress of emphysema; the mathematical model compares said angle with that of a control and/or the mathematical model compares automatically calculates expiratory airway collapse due to loss of alveolar attachments in emphysema.

The present invention also provides uses of the method or system according to the present invention for any of the following: to define emphysema or for diagnosing emphysema; to define the seriousness or progress of emphysema; to define a respiratory therapy medication; to define emphysema or for diagnosing emphysema without radiological imaging such as CT scanning; to define emphysema or for diagnosing emphysema whereby an angle smaller than 140° is indicative for emphysema; to define emphysema or for diagnosing emphysema whereby an angle smaller than 135° is indicative for emphysema; to define emphysema or for diagnosing emphysema whereby an angle smaller than 130° is indicative for emphysema; to define emphysema or for diagnosing emphysema whereby an angle smaller than 125° is indicative for emphysema and to define emphysema or for diagnosing emphysema whereby an angle smaller than 120° is indicative for emphysema.

The present invention also provides an apparatus for diagnosing emphysema, the apparatus comprising a) a device for receiving and sensing forced or total expiration of the respiratory system of a patient and further comprising b) a calculator or signal processor calculating the flow volume curve or relationship from said forced or total airway expiration, characterized in that the calculator or signal processor comprises 1) a first calculating means for calculating the flow-volume curve or the flow-volume relationship and 2) a second calculating means for automatically calculating the angle in the expiratory flow-volume curve between the two best fitting linear regression curves on flow-volume curve, whereby the signal processor or calculator further comprising a mathematical model to compare said angle with that of a COPD disorder patient subgroups or with a reference angle of one or more such COPD disorder subgroups.

The present invention further provides an apparatus for diagnosing emphysema whereby the apparatus having a) a signal input to receive electrical signals of an electrical signals produced by volume sensor and flow sensors of a device for receiving and sensing forced or total expiration of the respiratory system of a patient•and b) a calculator or signal processor calculating the flow volume curve or relationship from said forced or total airway expiration, characterized in that the calculator or signal processor comprises 1) a first calculating means for calculating the flow-volume curve or the flow-volume relationship and 2) a second calculating means for automatically calculating the angle in the expiratory flow-volume curve between the two best fitting linear regression curves on the flow-volume curve corresponding to entire exhalation flow rate and exhalation volume of a patients respiratory system, whereby the signal processor or calculator further comprising a mathematical model to compare said angle with that of a COPD disorder patient subgroups or with a reference angle of one or more such COPD disorder subgroups. These apparatuses can comprise a signal processor or calculator comprising a with mathematical model to compare said angle with that of plurality of chronic obstructive pulmonary disease (COPD) patients with no emphysema or with a reference angle of COPD but no emphysema.

In an embodiment of the apparatus the signal processor or calculator comprises a mathematical model to compare said angle with that of plurality of COPD patients affected with emphysema or with a reference angle for COPD and emphysema

In another embodiment of the apparatus the signal processor or calculator comprises a mathematical model to compare said angle with that of plurality of COPD patients affected with a defined seriousness or with defined progress of emphysema.

In another embodiment of the apparatus the signal processor or calculator comprises a mathematical model to compare said angle with that of a control.

Furthermore the apparatuses according to the present invention can be characterized in that the apparatus comprises a signal processor comprising a mathematical model that is described to automatically calculate expiratory airway collapse due to loss of alveolar attachments in emphysema.

It is an object of the present invention to provide such apparatus with a calculating means that is adapted for calculating the peak-to-surface area. This provides the surprising advantage that emphysema disorder can be distinguished in a in a chronic obstructive pulmonary disease (COPD) patient group by a device for receiving and sensing forced or total expiration of the respiratory system of a patient for instance by a spirometer. A simple, fast and accurate technology to discover emphysema disorder and advocate a proper corresponding treatment.

The present invention also provides uses of the an apparatuses according to present invention as described in this application here above to define emphysema or for diagnosing emphysema without radiological imaging such as CT scanning; to define emphysema or for diagnosing emphysema without radiological imaging such as CT scanning; to define the seriousness or progress of emphysema; to define a respiratory therapy medication; to define emphysema or for diagnosing emphysema whereby an angle smaller than 140° is indicative for emphysema; to define emphysema or for diagnosing emphysema whereby an angle smaller than 135° is indicative for emphysema; to any one of the claims 1 to 8, to define emphysema or for diagnosing emphysema whereby an angle smaller than 130° is indicative for emphysema; to define emphysema or for diagnosing emphysema whereby an angle smaller than 125° is indicative for emphysema and/or to define emphysema or for diagnosing emphysema whereby an angle smaller than 120° is indicative for emphysema.

What is claimed is:

1. A system for diagnosing a emphysema disorder in a chronic obstructive pulmonary disease (COPD) patient, the system comprising: 1) a sampling device to obtain total expiration or exhalation of the respiratory system of the subject; 2) a detection device generating flow and volume data of said total expiration or exhalation; and 3) a computer loaded a model a model to calculate the flow-volume loop; the two best fitting linear regression curves on flow-volume loop from peak flow to end of expiration and the angle between both regression lines and further loaded with a flow-volume loop reference profile for a emphysema disorder; wherein the computer receives and compares subject's flow-volume loop profile with the reference flow-volume loop profile.

2. A system for diagnosing a emphysema disorder in a chronic obstructive pulmonary disease (COPD) patient, the system comprising: a sampling device to obtain total expiration or exhalation of the respiratory system of the subject; a detection device generating flow and volume data of said total expiration or exhalation; and a computer loaded model to calculate the flow-volume loop; the two best fitting linear regression curves on flow-volume loop from peak flow to end of expiration and the angle between both regression lines and further loaded with a flow-volume loop reference profile for an emphysema disorder; wherein the computer receives and compares subject's flow-volume loop profile with the reference flow-volume loop profile.

3. A method of diagnosing an emphysema disorder in a subject with COPD, the method comprising: generating flow and volume data of total expiration or forced exhalation of a subject, calculating a profile of the two best fitting linear regression curves on flow-volume loop from peak flow and the angle between both regression lines, thereby providing; obtaining a reference profile for emphysema or for no emphysema; and comparing the subject profile with the reference profile, wherein a match of the subject profile to the reference profile indicates that the subject has emphysema or has no emphysema.

4. The method or system according to any of the previous claims 1 to 3, further comprising calculating means for calculating the peak-to-surface area.

5. The method or system according to any of the previous claims 1 to 4, whereby a mathematical model compares said angle with that of plurality of chronic obstructive pulmonary disease (COPD) patients with no emphysema

6. The method or system according to any of the previous claims 1 to 4, whereby a mathematical model compares said angle with that of plurality of COPD patients affected with emphysema

7. The method or system according to any of the previous claims 1 to 4, whereby a mathematical model compares said angle with that of plurality of COPD patients affected with a defined seriousness or with defined progress of emphysema.

8. The method or system according to any of the previous claims 1 to 4, whereby a mathematical model compares said angle with that of a control.

9. The method or system according to any of the previous claims 1 to 4, whereby a mathematical model compares automatically calculates expiratory airway collapse due to loss of alveolar attachments in emphysema.

10. The method or system according to any of the previous claims 1 to 4, to define the seriousness or progress of emphysema

11. The method or system according to any of the previous claims 1 to 4, to define a respiratory therapy medication

12. The method or system according to any of the previous claims 1 to 4, to define emphysema or for diagnosing emphysema without radiological imaging such as CT scanning.

13. The method or system according to any of the previous claims 1 to 4, to define emphysema or for diagnosing emphysema whereby an angle smaller than 140° is indicative for emphysema.

14. The method or system according to any of the previous claims 1 to 4, to define emphysema or for diagnosing emphysema whereby an angle smaller than 135° is indicative for emphysema

15. The method or system according to any of the previous claims 1 to 4, to define emphysema or for diagnosing emphysema whereby an angle smaller than 130° is indicative for emphysema

16. The method or system according to any of the previous claims 1 to 4, to define emphysema or for diagnosing emphysema whereby an angle smaller than 125° is indicative for emphysema

17. The method or system according to any of the previous claims 1 to 4, to define emphysema or for diagnosing emphysema whereby an angle smaller than 120° is indicative for emphysema

18. The use of the apparatus according to any one of the claims 1 to 9, to define emphysema or for diagnosing emphysema.

19. An apparatus for diagnosing emphysema, the apparatus comprising a) a device for receiving and sensing forced or total expiration of the respiratory system of a patient and further comprising b) a calculator or signal processor calculating the flow volume curve or relationship from said forced or total airway expiration, characterized in that the calculator or signal processor comprises 1) a first calculating means for calculating the flow-volume curve or the flow-volume relationship and 2) a second calculating means for automatically calculating the angle in the expiratory flow-volume curve between the two best fitting linear regression curves on flow-volume curve, whereby the signal processor or calculator further comprising a mathematical model to compare said angle with that of a COPD disorder patient subgroups or with a reference angle of one or more such COPD disorder subgroups.

20. An apparatus for diagnosing emphysema whereby the apparatus having a) a signal input to receive electrical signals of an electrical signals produced by volume sensor and flow sensors of a device for receiving and sensing forced or total expiration of the respiratory system of a patient and b) a calculator or signal processor calculating the flow volume curve or relationship from said forced or total airway expiration, characterized in that the calculator or signal processor comprises 1) a first calculating means for calculating the flow-volume curve or the flow-volume relationship and 2) a second calculating means for automatically calculating the angle in the expiratory flow-volume curve between the two best fitting linear regression curves on the flow-volume curve corresponding to entire exhalation flow rate and exhalation volume of a patients respiratory system, whereby the signal processor or calculator further comprising a mathematical model to compare said angle with that of a COPD disorder patient subgroups or with a reference angle of one or more such COPD disorder subgroups.

21. The apparatus according to any one of the previous claims 19 to 20, whereby the signal processor or calculator comprising a mathematical model to compare said angle with that of plurality of chronic obstructive pulmonary disease (COPD) patients with no emphysema or with a reference angle of COPD but no emphysema.

22. The apparatus according to any one of the previous claims 19 to 20, whereby the signal processor or calculator comprising a mathematical model to compare said angle with that of plurality of COPD patients affected with emphysema or with a reference angle for COPD and emphysema

23. The apparatus according to any one of the previous claims 19 to 20, whereby the signal processor or calculator comprising a mathematical model to compare said angle with that of plurality of COPD patients affected with a defined seriousness or with defined progress of emphysema.

24. The apparatus according to any one of the previous claims 19 to 20, whereby the signal processor or calculator further comprising a mathematical model to compare said angle with that of a control.

25. The apparatus according to any one of the previous claims 19 to 20, characterized in that the apparatus comprises a signal processor comprising a mathematical model that is described to automatically calculate expiratory airway collapse due to loss of alveolar attachments in emphysema.

26. The apparatus according to any one of the previous claims 19 to 20, further comprising calculating means for calculating the peak-to-surface area.

27. The apparatus according to any one of the previous claims 19 to 26, whereby the device for receiving and sensing forced or total expiration of the respiratory system of a patient is a spirometer.

28. The use of an apparatus for diagnosing emphysema according to any one of the previous claims 19 to 27, to define emphysema or for diagnosing emphysema without radiological imaging such as CT scanning.

29. The use of an apparatus for diagnosing emphysema according to any one of the previous claims 19 to 27, to define emphysema or for diagnosing emphysema.

30. The use of an apparatus for diagnosing emphysema according to any one of the previous claims 19 to 27, to define the seriousness or progress of emphysema

31. The use of an apparatus for diagnosing emphysema according to any one of the previous claims 19 to 27, to define a respiratory therapy medication

32. The use of an apparatus for diagnosing emphysema according to any one of the previous claims 19 to 27, to define emphysema or for diagnosing emphysema whereby an angle smaller than 140° is indicative for emphysema.

33. The use of an apparatus for diagnosing emphysema according to any one of the previous claims 19 to 27, to define emphysema or for diagnosing emphysema whereby an angle smaller than 135° is indicative for emphysema

34. The use of an apparatus for diagnosing emphysema according to any one of the previous claims 19 to 27, to define emphysema or for diagnosing emphysema whereby an angle smaller than 130° is indicative for emphysema

35. The use of an apparatus for diagnosing emphysema according to any one of the previous claims 19 to 27, to define emphysema or for diagnosing emphysema whereby an angle smaller than 125° is indicative for emphysema

36. The use of an apparatus for diagnosing emphysema according to any one of the previous claims 19 to 27, to define emphysema or for diagnosing emphysema whereby an angle smaller than 120° is indicative for emphysema
